(19) United States
(12) Patent Application Publication
(10) Pub. No.: US 2013/0323840 A1
(43) Pub. Date: Dec. 5, 2013

(54) CYLINDER CHANNEL HAVING A SAWTOOTH-SHAPED CROSS SECTION, COAXIAL CHANNEL INCLUDING SAME AND METHOD FOR MANUFACTURING SAME

(71) Applicants: Sang Hoon Lee, Gyeonggi-do (KR); Edward Kang, Gyeonggi-do (KR)

(72) Inventors: Sang Hoon Lee, Gyeonggi-do (KR); Edward Kang, Gyeonggi-do (KR)

(73) Assignee: KOREA UNIVERSITY RESEARCH AND BUSINESS FOUNDATION, Seoul (KR)

(21) Appl. No.: 13/996,961

(22) PCT Filed: Dec. 19, 2011
(86) PCT No.: PCT/KR2011/009780

§ 371 Date: Aug. 14, 2013

(30) Foreign Application Priority Data
Dec. 24, 2010 (KR) 10-2010-0134507

(51) Int. Cl. B81B 1/00; C12N 5/079; B81C 1/00

(52) U.S. Cl. CPC B81B 1/002; B81C 1/00634; C12N 5/0618
USPC 435/398; 428/34.1; 264/320; 156/245; 264/328.1

(57) ABSTRACT

The present disclosure relates to a cylinder channel having a sawtooth-shaped cross section, a method for manufacturing same, a coaxial channel including same and a method for manufacturing a microfiber or a microparticle having a sawtooth-shaped cross section using same.

### TECHNICAL FIELD

The present disclosure relates to a relates to a cylinder channel having a sawtooth-shaped cross section, a method for manufacturing same, a coaxial channel including same and a method for manufacturing a microfiber or a microparticle having a sawtooth-shaped cross section using same.

### BACKGROUND ART

Many biochips and microchips have been developed based on the micro technology. Among them, microfluidic chips are used extensively in cell researches and chemical engineering researches and the application is extending toward sensors, cell culture chips, tissue production, or the like. As the use of the microfluidic chips increases, the shape of the channels of the microchips becomes more complicated and diverse. Accordingly, to provide better research environment, it is of great importance to provide 3-dimensional channels having various cross sections.

Basically, since the channels are mostly manufactured in top-down or down-top fashion, microchannels having only round, tetragonal or polygonal cross sections can be manufactured with the current technique. Accordingly, there is a need to develop channels having more complicated and various shapes for wider application of microchips.

### DISCLOSURE

### Technical Problem

The present disclosure is directed to providing a method for manufacturing a cylinder channel having a sawtooth-shaped cross section (hereinafter, also referred to as ‘sawtooth groove’), a method for manufacturing a coaxial channel including the cylinder channel and a method for manufacturing a microfiber having an opposite sawtooth-shaped cross section using same.

### Technical Solution

In an aspect, the present disclosure provides a cylinder channel having a sawtooth-shaped cross section.

In another aspect, the present disclosure provides a method for manufacturing a cylinder channel having a tapered sawtooth-shaped cross section.

In another aspect, the present disclosure provides a molded part of a cylinder channel having a sawtooth-shaped cross section prepared by the above-described method.

In another aspect, the present disclosure provides a coaxial channel including a cylinder channel having a sawtooth-shaped cross section.

In another aspect, the present disclosure provides a method for manufacturing a coaxial channel including a cylinder channel having a sawtooth-shaped cross section.

In another aspect, the present disclosure provides a molded part including a coaxial channel including a cylinder channel having a sawtooth-shaped cross section which is prepared by the above-described method.

In another aspect, the present disclosure provides a method for manufacturing a microfiber having a sawtooth-shaped cross section using the microfluidic chip including the coaxial channel including the cylinder channel having a sawtooth-shaped cross section and a method for aligning cells using same.

In another aspect, the present disclosure provides a method for manufacturing a microparticle having a sawtooth-shaped cross section using the coaxial channel including the cylinder channel having a sawtooth-shaped cross section.

The fiber and particle synthesized according to present disclosure will find various applications in the fields of biomedicine, tissue engineering and drug delivery.

### Advantageous Effects

The present disclosure allows development of various microfluidic chips by diversifying the kinds of microfluidic channels by providing a cylinder channel having a sawtooth-shaped cross section.

Further, the present disclosure allows improvement of the performance of a mixer or a blender by inducing an unstable fluid flow by modifying the angle of the sawtooth-shaped groove of a cylinder channel.

A microfiber having a sawtooth-shaped cross section manufactured using the cylinder channel having a sawtooth-shaped cross section according to the present disclosure may have enhanced strength.

Using the microfiber having a sawtooth-shaped cross section according to the present disclosure, it is possible to align tissues (or fibrils) and also to directly align cells on the channel.

Also, fibers of various shapes and functions can be manufactured using patterned coaxial channels.

### DESCRIPTION OF DRAWINGS

FIG. 1 a shows an actually manufactured microfluidic chip.

FIG. 1b shows production of a fiber by a spider.

FIG. 2 a schematically shows a method for manufacturing a coaxial channel including a cylinder channel having a sawtooth-shaped pattern.

FIG. 2 b is an SEM image of a fiber manufactured using a coaxial channel including a cylinder channel having a sawtooth-shaped cross section.

FIG. 2 c shows a result of testing strength of a fiber manufactured according to the present disclosure and a fiber without a sawtooth-shaped pattern.

FIG. 3 schematically shows a method for manufacturing a molded part of a coaxial channel according to a different method.

FIG. 4 a is a top view of a cylinder channel having a sawtooth-shaped sawtooth groove pattern according to the present disclosure which is bonded to a coaxial channel.

FIG. 4 b is a side view of a cylinder channel having a sawtooth-shaped sawtooth groove pattern according to the present disclosure which is bonded to a coaxial channel.

FIG. 4 c is an optical microscopic image of a master mold according to the present disclosure.

FIG. 5 a is an electron microscopic image of a fiber manufactured according to the present disclosure and FIG. 5 b is an electron microscopic image of a spider silk produced by a spider.

FIG. 6 is an SEM image of a fiber having ridges and having a sawtooth-shaped cross section, which is manufactured using a chip and a valve.

FIG. 8 shows a method for aligning cells using a fiber having a groove.

FIG. 9 shows fluorescence images obtained after culturing nerve cells for 5 days on the surface of a microfiber having grooves (right) and a smooth general fiber (left).

FIG. 10 shows fluorescence images obtained after culturing nerve cells for 5 days on the surface of a microfiber having grooves (left) and a smooth general fiber (middle) and a result of measuring angles between the cells and the groove (right). In the graph on the right side, angles whose absolute values are close to 0 mean that the cells are parallel to the groove.

### BEST MODE

As described above, microfibers having only round or polygonal cross sections can be manufactured with the current technique and a microfiber having a sawtooth-shaped cross section cannot be manufactured.

Accordingly, the present disclosure provides a cylinder channel having a sawtooth-shaped cross section to obtain fibers similar to spider silk.

Hereinafter, various aspects and embodiments of the present disclosure will be described in detail.

In an aspect, the present disclosure provides a cylinder channel of a microfluidic chip including a plurality of sawtooth grooves having a tapered or regular sawtooth-shaped cross section.

Specifically, the sawtooth groove may have a thickness of 3-15 μm and a width of 5-15 μm and the gap between the sawtooth grooves may be 10-20 μm. Outside this range, it is difficult to obtain a microfiber or microparticle desired by the present disclosure.

In another aspect, the present disclosure provides a method for manufacturing a cylinder channel, including positioning and bonding a membrane including a sawtooth groove on a base mold having a mold groove formed on an upper portion thereof and a hole for pressure control formed on a lower portion thereof such that the membrane including the sawtooth groove faces upward and controlling pressure such that the pressure at the upper portion of the membrane is lower than the lower portion and the membrane is deformed toward the lower portion at the mold groove, wherein the membrane has a plurality of sawtooth grooves having a sawtooth-shaped cross section at the upper portion and said controlling of the pressure is carried out using the hole for pressure control.

In the present disclosure, the deformation of the membrane means a deformation whereby the membrane is deformed to have a naturally curved surface by pressure difference as a result of the pressure control by the hole for pressure control at the lower portion of the base mold.

In another aspect, the present disclosure provides a method for manufacturing a coaxial channel including the cylinder channel having a sawtooth groove, including: 1) forming a mold layer on a wafer and forming a plurality of sawtooth grooves on an upper portion of the mold layer using a photoresist; 2) coating a membrane on the upper portion of the mold layer and curing same to obtain a membrane having a sawtooth-shaped surface; 3) bonding a base mold having a mold groove formed on an upper portion thereof and a hole for pressure control formed on a lower portion thereof on the membrane and positioning the membrane such that the plurality of sawtooth grooves of the membrane face upward; 4) controlling pressure using the hole for pressure control such that the pressure at the upper portion of the membrane is lower than the lower portion and the membrane is deformed toward the lower portion at the mold groove; 5) positioning a photosensitive material on the deformed membrane, positioning a light-transmitting material on the photosensitive material and irradiating light onto the light-transmitting material to prepare a master mold including the photosensitive material; 6) preparing a molded part including a semicylindrical channel having a sawtooth-shaped cross section using the master mold; and 7) bonding two molded parts each including a semicylindrical channel having a sawtooth-shaped cross section to prepare a molded part including a cylinder channel having a sawtooth-shaped cross section.

In the method for manufacturing a coaxial channel according to the present disclosure, in 1), the thickness of the mold layer is not particularly limited but may be specifically 1-60 μm. The sawtooth-shaped sawtooth groove may have a thickness of 3-15 μm and a width of 5-15 μm and the gap between the sawtooth grooves may be 10-20 μm. Outside this range, it is impossible to manufacture a fiber having a desired sawtooth-shaped cross section.

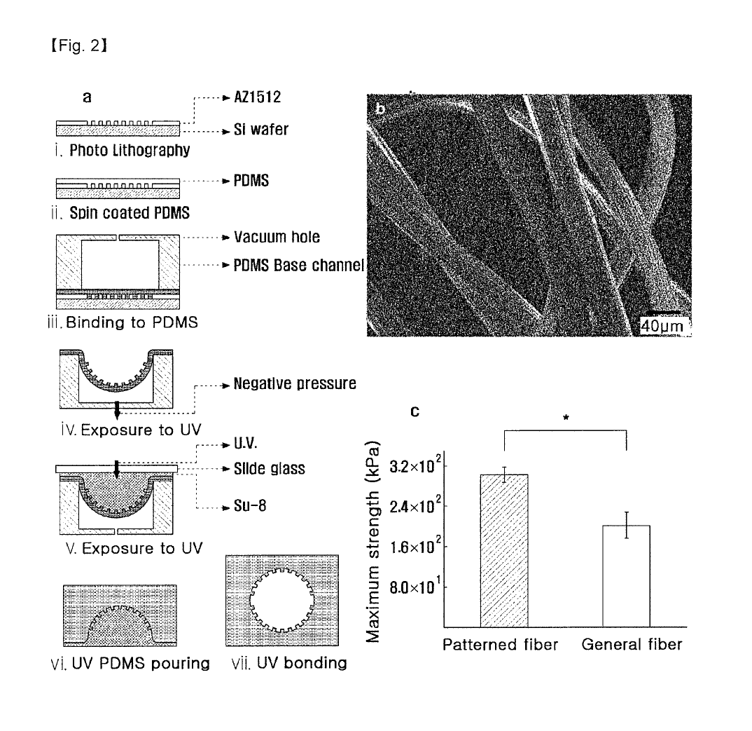

In 2), the sawtooth-shaped surface may be formed by a photolithography process using a fine sawtooth groove pattern and a positive photoresist (see FIG. 2 a i).

In 2), specifically, the membrane may be an elastomer selected from polydimethylsiloxane (PDMS), rubber, polybutadiene, polyisobutylene, polyurethane, etc., but is not limited thereto.

The membrane may be any one as long as it has a molecular weight in the range commonly used in the art to which the present disclosure belongs.

In 2), the thickness of the membrane may be selected appropriately in order to achieve the effect desired by the present disclosure. Specifically, a thickness of 1-60 μm may be desired to effectively achieve the effect desired by the present disclosure.

In 3), the base mold may be selected from PDMS, PMMA, plastic and cast metal such as gold or iron, but is not limited thereto.

In 3), the base mold having the mold groove formed on the upper portion thereof and the hole for pressure control formed on the lower portion thereof is bonded on the membrane and, after the mold layer formed in 1) is separated from the wafer by removing using a solvent, the membrane is positioned such that the sawtooth-shaped pattern faces upward.

In 3), the mold groove of the base mold may have a tapered or regular cross section and the base mold may be positioned on the membrane so as to form a coaxial channel according to the present disclosure.

In 4), the pressure control may be performed using the hole for pressure control such that the membrane is deformed toward the lower portion. The pressure control may be performed continuously or intermittently with time intervals.

In 5), the photosensitive material may be selected from SU-8, AZ PR and Norland Optical Adhesive (NOA), although not being limited thereto. More specifically, it may be SU-8. The light-transmitting material may be selected from glass, quartz, plastic, polystyrene, polyethylene, etc., although not being limited thereto. More specifically, it may be glass or quartz.

The SU-8 refers to a material having the following chemical formula.

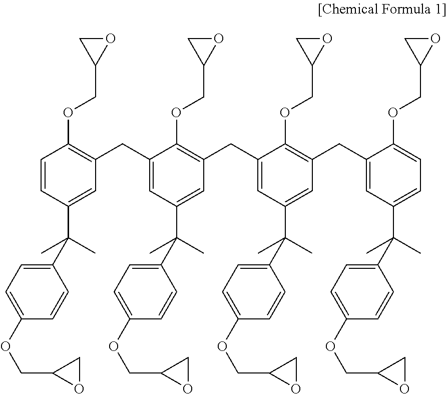 [SMILES: CC(C)(c1ccc(OCC2CO2)cc1)c1ccc(OCC2CO2)c(Cc2cc(C(C)(C)c3ccc(OCC4CO4)cc3)cc(Cc3cc(C(C)(C)c4ccc(OCC5CO5)cc4)cc(Cc4cc(C(C)(C)c5ccc(OCC6CO6)cc5)ccc4OCC4CO4)c3OCC3CO3)c2OCC2CO2)c1]

The photosensitive material or the light-transmitting material may be any one as long as it has a molecular weight in the range commonly used in the art to which the present disclosure belongs.

The light may be UV or visible light. Specifically, the effect desired by the present disclosure may be achieved effectively when the light is UV.

In 6), the molded part including the semicylindrical channel may be selected from PDMS, NOA, PMMA and acryl, but is not limited thereto.

In 7), the bonding of the two molded parts each including a semicylindrical channel having a sawtooth-shaped cross section may be performed using oxygen plasma, but is not limited thereto.

In another aspect, the present disclosure provides a coaxial channel including a cylinder channel having a sawtooth-shaped cross section, which includes (A) a main channel, (B) a sample channel and (C) one or more external channel. At least one of the main channel, the sample channel and the one or more external channel may be a cylinder channel having a circular or oval cross section. A terminal end of the sample channel may be connected to an initial end of the main channel. (i) The sample channel may be tapered toward the terminal end portion or (ii) only the terminal end portion of the sample channel may be tapered toward the portion connected with the main channel and the remaining portion may be constant in size and shape of the cross section, with at least a part of the sample channel having a sawtooth-shaped cross section. The one or more external channel may be connected to a side of the main channel.

In the present disclosure, the initial end and the terminal end of the channel refer to an end portion where the flow of a medium in the channel begins and an end portion where the flow of the medium ends, respectively.

The main channel is connected to the sample channel and the external channel. A material flowing out of the sample channel enters the main channel, is cured after meeting with a material flowing out of the external channel and is discharged as a microfiber or a microparticle.

The sample channel refers to a channel through which a sample (fluid) flows. And, the external channel refers to a channel which is connected to the side of the main channel and through which a material that cures a sample flowing out of the sample channel passes.

The coaxial channel (i) may be constant in size along a longitudinal direction, (ii) may decrease or increase linearly in size along the longitudinal direction or (iii) may be a combination of thereof.

The sample channel may be tapered toward the terminal end portion and the remaining portion may be constant in size and shape of the cross section, with at least a part of the sample channel having a sawtooth-shaped cross section.

A longitudinal axis in the main channel may be in line with a longitudinal axis in the sample channel, a longitudinal axis in the external channel may cross with a longitudinal axis in the main channel, and all the longitudinal axes in the main channel, the sample channel and the one or more external channel may be in the same plane. In this case, the effect desired by the present disclosure may be achieved effectively.

In another aspect, the present disclosure provides a molded part including the cylinder channel having a sawtooth-shaped cross section according to the present disclosure. Any molded part satisfying the structure and function described above is included in the scope of the present disclosure. However, a molded part manufactured according to the method of the present disclosure is advantageous over a molded part manufactured according to a different method in that the effect desired by the present disclosure can be achieved more effectively.

In another aspect, the present disclosure provides a method for manufacturing a microfiber having a sawtooth-shaped cross section using the coaxial channel including the cylinder channel having a sawtooth-shaped cross section, which includes: (A) injecting a sample material into the sample channel; and (B) injecting an external material into the external channel.

Specifically, (A) and (B) may be carried out simultaneously. Alternatively, they may be carried out sequentially, continuously or intermittently with time interval.

In another exemplary embodiment, the sample material may be (i) a non-UV-curable material selected from PLGA, alginate, chitosan and collagen, (ii) a UV-curable material selected from 4-HBA, PNIPAAM, Norland Optical Adhesive (NOA) and PEG or (iii) a mixture thereof. The mixing ratio is not particularly limited but may be 1:9-9:1.

The content of the sample material is not particularly limited. For example, the content of the sample material may be 1-5 wt % and the content of a solvent may be 95-99 wt %. If the content of the sample material is less than 1 wt %, a microfiber may not be manufactured. And, if the content of the sample material exceeds 5 wt %, the sample material may not be dissolved.

The solvent is not particularly limited. For example, water, brine, cell culture medium, etc. may be used.

In another exemplary embodiment, the external material may be a solution wherein (i) a first external material selected from calcium chloride, sodium chloride and a mixture thereof is dissolved in (ii) a second external material selected from water, cell culture, PBS and a mixture thereof. In this case, the effect desired by the present disclosure can be achieved effectively.

Specifically, the external material may include 1-5 wt % of the first external material and 95-99 wt % of the second external material. If the content of the first external material is less than 1 wt %, the microfiber may not be cured. And, if the content of the second external material exceeds 5 wt %, the first external material may not be dissolved.

In another aspect, the present disclosure provides a method for controlling the diameter of a microfiber having a sawtooth-shaped cross section manufactured using the coaxial channel including the cylinder channel having a sawtooth-shaped cross section according to the present disclosure, which includes controlling (i) the injection rate of the sample material into the sample channel and (ii) the injection rate of the external material into the external channel.

In an exemplary embodiment, (i) the injection rate of the sample material into the sample channel may be controlled in the range of 0.6-1.8 mL/h and (ii) the injection rate of the external material into the external channel may be controlled in the range of 20-40 mL/h. In this case, the effect desired by the present disclosure can be achieved effectively.

In another aspect, the present disclosure provides a method for manufacturing a microparticle having a sawtooth-shaped cross section using the coaxial channel including the cylinder channel having a sawtooth-shaped cross section, which includes: (A) injecting a sample material into the sample channel; and (B) injecting an external material into the external channel.

Specifically, (A) and (B) may be carried out simultaneously. Alternatively, they may be carried out sequentially, continuously or intermittently with time interval.

In another exemplary embodiment, the sample material may be (i) a non-UV-curable material selected from PLGA, alginate, chitosan and collagen, (ii) a UV-curable material selected from 4-HBA, PNIPAAM, NOA and PEG or (iii) a mixture thereof. The mixing ratio is not particularly limited but may be 1:9-9:1.

The content of the sample material is not particularly limited. For example, the content of the sample material may be 1-5 wt % and the content of a solvent may be 95-99 wt %. If the content of the sample material is less than 1 wt %, a microfiber may not be manufactured. And, if the content of the sample material exceeds 5 wt %, the sample material may not be dissolved.

The solvent is not particularly limited. For example, water, brine, cell culture medium, etc. may be used.

In another exemplary embodiment, the external material may be a solution wherein (i) a first external material selected from calcium chloride, sodium chloride, etc. is dissolved in (ii) a second external material selected from organic solvents such as oleic acid, soybean oil, methanol, dodecane, etc.

In particular, when the external material is prepared by: (a) preparing a first external material solution by dissolving the first external material in (iii) a third external material selected from 2-methyl-1-propanol, isopropyl alcohol and a mixture thereof; (b) preparing a mixture solution by mixing the first external material solution with the third external material; and (c) distilling the mixture solution, the effect desired by the present disclosure can be achieved effectively.

The external material may include 1-5 wt % of the first external material alone or a mixture thereof with the second external material and 95-99 wt % of the third external material. If the content of the first external material alone or the mixture thereof with the second external material is less than 1 wt %, the microfiber may not be cured. And, if the content of the first external material alone or the mixture thereof with the second external material exceeds 5 wt %, the first external material alone or the mixture thereof with the second external material may not be dissolved.

In another aspect, the present disclosure provides a method for controlling the diameter of a microparticle having a sawtooth-shaped cross section manufactured using the coaxial channel including the cylinder channel having a sawtooth-shaped cross section according to the present disclosure, which includes controlling (i) the injection rate of the sample material into the sample channel and (ii) the injection rate of the external material into the external channel.

In an exemplary embodiment, (i) the injection rate of the sample material into the sample channel may be controlled in the range of 0.6-1.8 mL/h and (ii) the injection rate of the external material into the external channel may be controlled in the range of 5-35 mL/h. In this case, the effect desired by the present disclosure can be achieved effectively.

### Mode for Invention

Hereinafter, the present disclosure will be described in detail through examples. However, the following examples are for illustrative purposes only and it will be apparent to those of ordinary skill in the art that the scope of the present disclosure is not limited by the examples.

### EXAMPLES

### Manufacturing of PDMS Coaxial Channel

Manufacturing of Coaxial Channel Including Cylinder Channel Having Sawtooth-Shaped Cross Section

In order to manufacture a cylinder channel having a sawtooth-shaped cross section, a mold layer was formed on a silicon wafer and a part of the mold layer was patterned into fine sawtooth grooves by a photolithography process (FIG. 2 a i) using AZ 1512 photoresist as shown in FIG. 2. The sawtooth grooves were patterned on the mold layer with a thickness of 10 μm, a width of 15 μm and a gap between the sawtooth grooves of 10 μm. On top of the patterned mold, a replicated membrane was prepared by spin-coating a PDMS membrane with a thickness of 10 μm (FIG. 2 a ii).

A quadrangular PDMS membrane channel having a degassing port (hole for pressure control) was bound to the replicated membrane using oxygen plasma and the mold was removed using acetone to prepare a channel wherein the replicated membrane was separated from the Si wafer and bound to the quadrangular PDMS membrane channel (FIG. 2 a iii)). Next, the channel wherein the replicated membrane was bound to the quadrangular PDMS membrane channel was positioned such that the sawtooth-shaped membrane faced upward and a concave semicylindrical channel structure was formed by controlling pressure using the hole for pressure control (FIG. 2 a iv)). After pouring SU-8 into the deformed semicylindrical membrane and covering with glass, the deformed membrane was cured by irradiating UV (FIG. 2 a v)). Then, an SU-8 master mold of a coaxial channel having a sawtooth-shaped semicylindrical channel was obtained by separating from the quadrangular PDMS membrane channel (FIG. 2 a vi)). The SU-8 master mold was in the form of a coaxial channel according to the present disclosure and included a main channel, two, four or six sample channels and one or two external channels. Liquid PDMS was poured into the master mold and cured in an oven to obtain a molded part of a PDMS cylinder channel having a sawtooth-shaped cross section. Thus obtained two molded parts of a PDMS cylinder channel were bonded using oxygen plasma to prepare a coaxial channel including a cylinder channel having a sawtooth-shaped cross section.

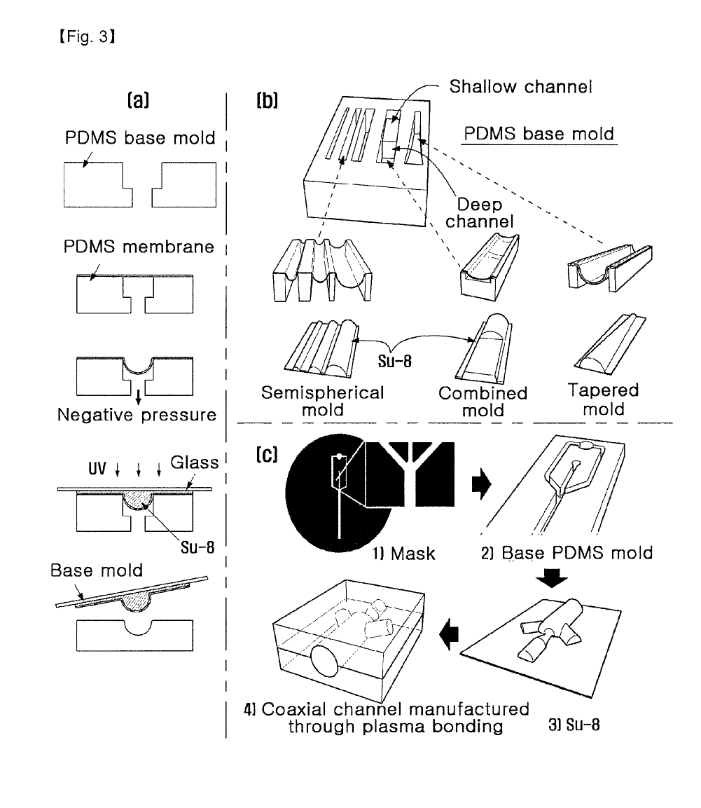

The coaxial channel included a main channel, two sample channels and one or more external channels and had various shapes and dimensions such as pseudo-rectangular structure, combined structure, tapered structure and coaxial structure as shown in FIG. 3b.

For example, the combined structure was formed using base molds having different depths. The shallow portion has a pseudo-rectangular shape as the deformed membrane spreads out at the bottom of the channel, whereas the membrane is deformed to form a cylindrical structure at the deep portion.

<Manufacturing of Spider Mimicking Chip (Fiber Generating Chip)>

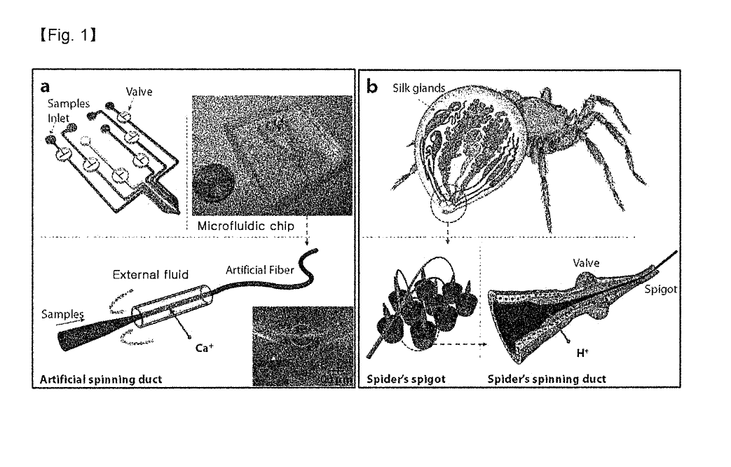

As an example of a microfluidic chip, a spider mimicking chip capable of producing microfibers of various shapes and functions was manufactured. FIG. 1 b shows production of spider silk by a spider. The actual spider produces silk from spigots through the spinning duct.

The spider mimicking chip has a thin sample injecting channel portion that produces a microfiber from different samples (artificial spigot).

FIG. 1 a shows a microfluidic chip manufactured based on the same principle.

Referring to FIG. 1 a, the coaxial channel consists of a sample channel, a main channel and an external channel. The upper portion of the coaxial channel includes a portion where an alginate sample is injected (inlet of the sample channel) and a portion where a CaCl₂ solution is injected (inlet of the external channel). An electron microscopic image of the main channel and a process of preparing a fiber are shown at the bottom of FIG. 1 a. The alginate sample which flows out of the sample channel and enters the main channel is hardened into a gel by meeting with Ca²⁺ that flows out of the external channel. As a result, a single strand of hydrogel fiber is obtained.

<Generation of Fiber Using Coaxial Channel Including Cylinder Channel Having Sawtooth-Shaped Cross Section>

The scanning electron microscopic (SEM) image at the right top of FIG. 2 shows a fibrous scaffold having a sawtooth-shaped sawtooth groove pattern manufactured using the coaxial channel including the cylinder channel having a sawtooth-shaped cross section. The sawtooth groove patterned fiber was produced using the coaxial channel including the cylinder channel having a sawtooth-shaped cross section.

To prepare the fiber having sawtooth grooves, 3 wt % alginate aqueous solution and 2.8 wt % calcium chloride aqueous solution were used. The injection rate of the alginate solution and the calcium chloride solution was set at 1.3 mL/h and 23 mL/h, respectively, to decrease the gelling time of the alginate solution.

The strength of the obtained fiber having sawtooth grooves was tested as follows. The fiber was connected to a 0.1-g basket (aluminum, DuPont) and water was added to the basket in 0.1-g increments until the fiber was snapped.

As seen from FIG. 2 c, the fiber having sawtooth grooves exhibited 30% improved final strength as compared to a general fiber (without sawtooth grooves) under the same condition.

And, when two fibers of the same thickness were fixed and pulled downward, the patterned fiber exhibited about 1.5 times stronger ultimate strength as compared to the general fiber without patterns.

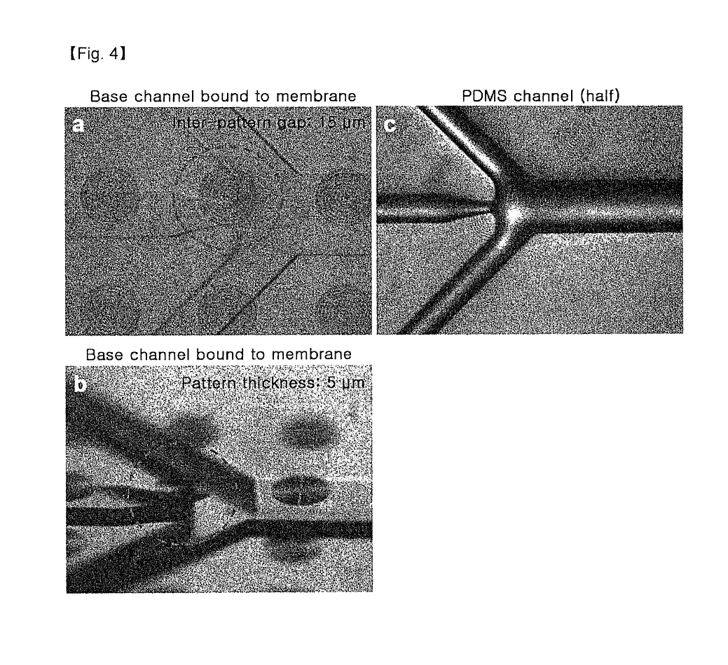

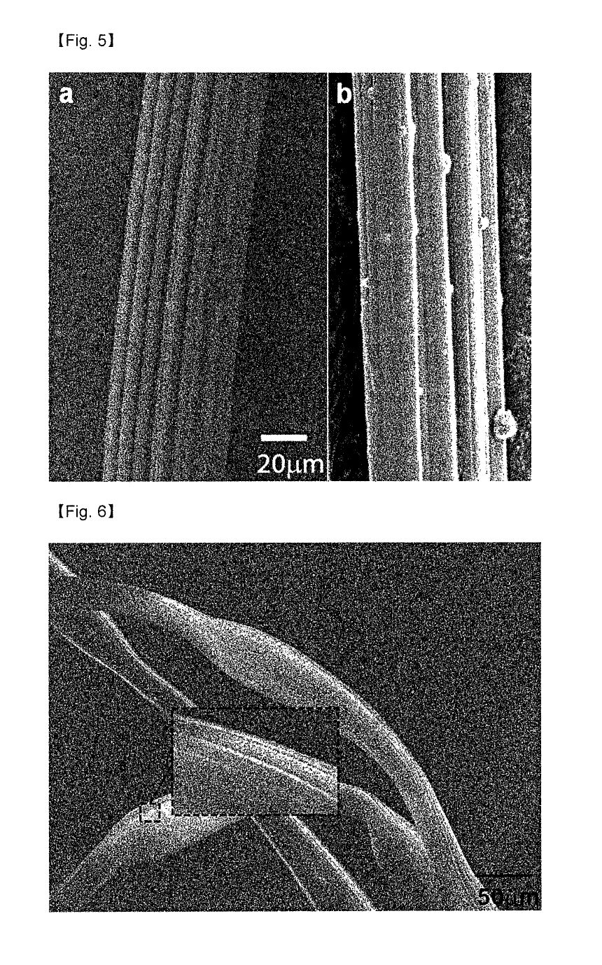

This result can be compared to the I-beam exhibiting better strength than a general beam in civil engineering or construction. FIG. 4 shows microscopic images of a patterned membrane having sawtooth grooves bound to a coaxial channel. FIG. 4 a is a top view of the membrane having sawtooth grooves bound to the coaxial channel. As seen from FIG. 4 a, the gap between the sawtooth-shaped sawtooth grooves is 15 μm. FIG. 4 b is a side view of the membrane having sawtooth grooves bound to the coaxial channel. As seen from FIG. 4 b, the sawtooth-shaped sawtooth groove pattern has a thickness of 5 μm. A fiber is formed at the portion where three channels converge. Accordingly, to prepare a sawtooth-shaped fiber, the respective channels should be aligned in parallel (see the red circle in FIG. 4 a). FIG. 4 c is an optical microscopic image of a master mold according to the present disclosure. It can be seen that the master mold has regular patterns formed by the sawtooth grooves of the PDMS membrane. FIG. 5 shows an electron microscopic image of a microfiber manufactured according to the present disclosure (a) and a spider silk produced by a spider (b). Whereas the spider silk consists of several threads of thin spider silk, the fiber manufactured according to the present disclosure is one thick fiber having sawtooth-shaped regular patterns. However, the two fibers look very similar.

<Alignment of Cells for Reconstruction of Muscle Tissue>

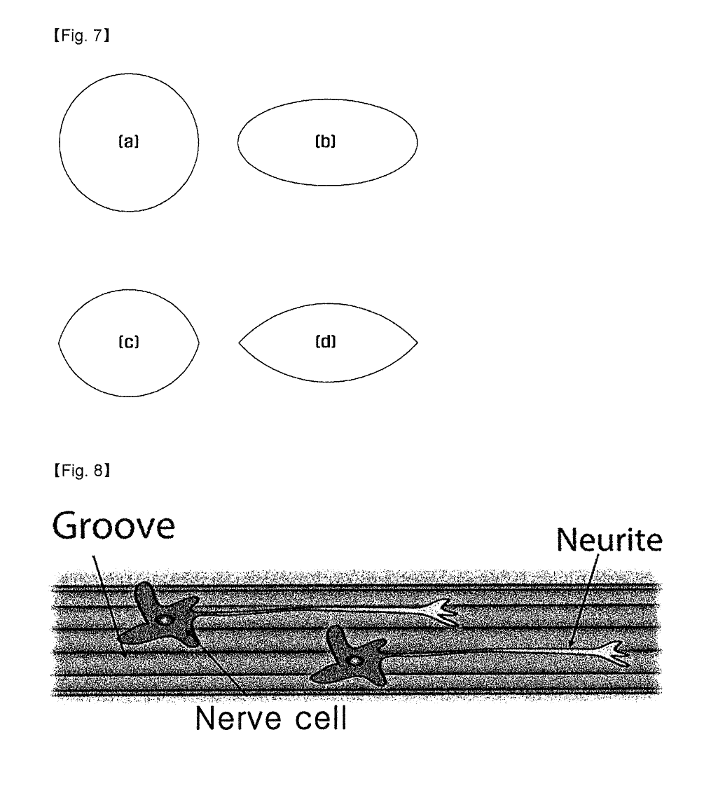

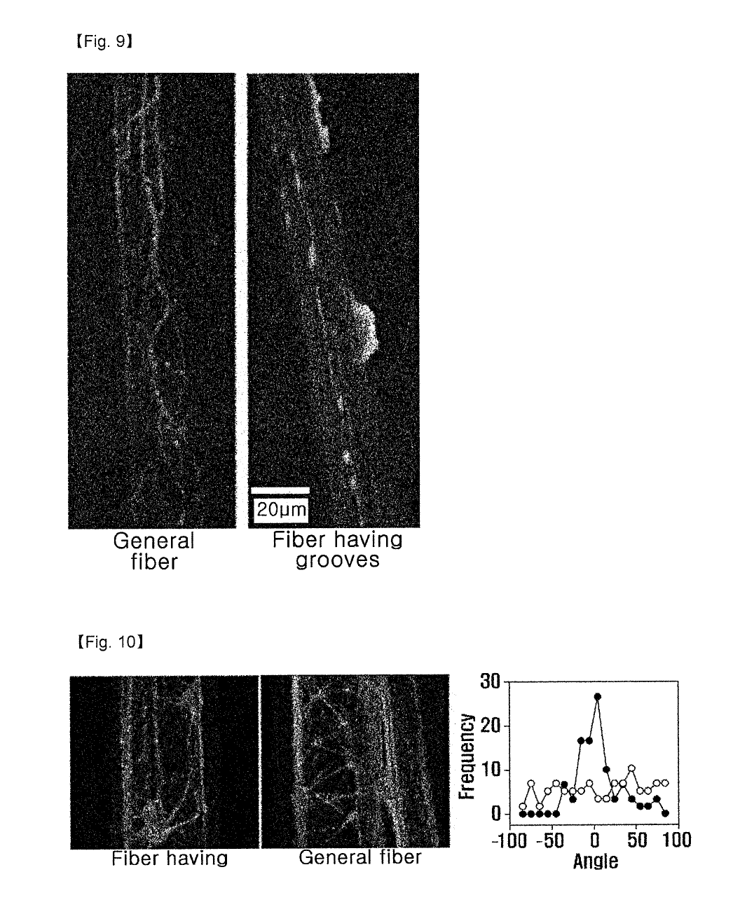

The technical feature of the present disclosure may be utilized to align cells by forming fine patterned grooves in the order of 2-10 μm on a microfiber, as shown in FIG. 8. For example, when nerve cells or muscle cells are cultured on a patterned microfiber, the cells grow along the grooves and are connected to other cells. FIG. 9 shows fluorescence images obtained after culturing nerve cells for 5 days on the surface of microfibers with or without grooves. As seen from FIG. 9, the nerve cells grew along the grooves and were connected to other cells.

### INDUSTRIAL APPLICABILITY

A result of measuring angles between the cells and the groove is shown in FIG. 10. It can be seen that the cells that grew on the patterned fiber are aligned almost in parallel with the grooves. In transplantation of spinal nerves or muscles, it is very important to keep them parallel to the scaffold to ensure intercellular signaling. Since an artificial tissue prepared using the microfiber having grooves according to the present disclosure allows regular alignment of cells and shortest connection between cells, it can be very useful in clinical applications.

1. A cylinder channel comprising a plurality of sawtooth grooves having a tapered or regular sawtooth-shaped cross section in a longitudinal direction on the surface thereof.

2. The cylinder channel according to claim 1, wherein the sawtooth groove has a thickness of 3-15 μm and a width of 5-15 μm and the gap between the sawtooth grooves is 10-20 μm.

3. A method for manufacturing a cylinder channel, comprising positioning a membrane on a base mold having a mold groove formed on an upper portion thereof and a hole for pressure control formed on a lower portion thereof and controlling pressure such that the pressure at the upper portion of the membrane is lower than the lower portion and the membrane is deformed toward the lower portion at the mold groove, wherein the membrane has a plurality of sawtooth grooves having a sawtooth-shaped cross section at the upper portion and said controlling of the pressure is carried out using the hole for pressure control.

4. A method for manufacturing a coaxial channel comprising the cylinder channel having a sawtooth groove having a tapered or regular sawtooth-shaped cross section in a longitudinal direction on the surface thereof, comprising: 1) forming a mold layer on a wafer and forming a plurality of sawtooth grooves on an upper portion of the mold layer using a photoresist; 2) coating a membrane on the upper portion of the mold layer and curing same to obtain a membrane having a sawtooth-shaped surface; 3) bonding a base mold having a mold groove formed on an upper portion thereof and a hole for pressure control formed on a lower portion thereof on the membrane and positioning the membrane such that the plurality of sawtooth grooves of the membrane face upward; 4) controlling pressure using the hole for pressure control such that the pressure at the upper portion of the membrane is lower than the lower portion and the membrane is deformed toward the lower portion at the mold groove; 5) positioning a photosensitive material on the deformed membrane, positioning a light-transmitting material on the photosensitive material and irradiating light onto the light-transmitting material to prepare a master mold comprising the photosensitive material; 6) preparing a molded part comprising a semicylindrical channel having a sawtooth-shaped cross section using the master mold; and 7) bonding two molded parts each comprising a semicylindrical channel having a sawtooth-shaped cross section to prepare a molded part comprising a cylinder channel having a sawtooth-shaped cross section.

5. The method for manufacturing a coaxial channel according to claim 4, wherein the sawtooth groove has a thickness of 3-15 μm and a width of 5-15 μm, the gap between the sawtooth grooves is 10-20 μm, the sawtooth groove is formed by a photolithography process using a fine sawtooth groove pattern and a positive photoresist, the mold groove of the base mold has a tapered or regular cross section, the membrane is selected from polydimethylsiloxane, rubber, polybutadiene, polyisobutylene and polyurethane and has a thickness of 1-60 μm, the base mold is selected from PDMS, PMMA, plastic and cast metal, the photosensitive material is selected from SU-8, AZ PR and NOA, the light-transmitting material is selected from glass, quartz, plastic, polystyrene and polyethylene, the molded part comprising the semicylindrical channel is selected from PDMS, NOA, PMMA and acryl, and the bonding of the two molded parts each comprising the semicylindrical channel is performed using oxygen plasma.

6. A molded part comprising a coaxial channel comprising a cylinder channel having a sawtooth-shaped cross section, which is manufactured by
1) forming a mold layer on a wafer and forming a plurality of sawtooth grooves on an upper portion of the mold layer using a photoresist; 2) coating a membrane on the upper portion of the mold layer and curing same to obtain a membrane having a sawtooth-shaped surface; 3) bonding a base mold having a mold groove formed on an upper portion thereof and a hole for pressure control formed on a lower portion thereof on the membrane and positioning the membrane such that the plurality of sawtooth grooves of the membrane face upward; 4) controlling pressure using the hole for pressure control such that the pressure at the upper portion of the membrane is lower than the lower portion and the membrane is deformed toward the lower portion at the mold groove; 5) positioning a photosensitive material on the deformed membrane, positioning a light-transmitting material on the photosensitive material and irradiating light onto the light-transmitting material to prepare a master mold comprising the photosensitive material; 6) preparing a molded part comprising a semicylindrical channel having a sawtooth-shaped cross section using the master mold; and 7) bonding two molded parts each comprising a semicylindrical channel having a sawtooth-shaped cross section to prepare a molded part comprising a cylinder channel having a sawtooth-shaped cross section.

7. A coaxial channel comprising a cylinder channel having a sawtooth-shaped cross section, which comprises (A) a main channel, (B) a sample channel and (C) one or more external channel, wherein at least one of the main channel, the sample channel and the one or more external channel is a cylinder channel having a circular or oval cross section, a terminal end of the sample channel is connected to an initial end of the main channel, (i) the sample channel is tapered toward the terminal end portion or (ii) only the terminal end portion of the sample channel is tapered toward the portion connected with the main channel and the remaining portion is constant in size and shape of the cross section, with at least a part of the sample channel having a sawtooth-shaped cross section, and the one or more external channel is connected to a side of the main channel.

8. The coaxial channel comprising a cylinder channel having a sawtooth-shaped cross section according to claim 7, wherein the coaxial channel (i) is constant in size along a longitudinal direction, (ii) decreases or increases linearly in size along the longitudinal direction or (iii) is a combination of thereof, the sample channel is tapered toward the terminal end portion and the remaining portion is constant in size and shape of the cross section, with at least a part of the sample channel having a sawtooth-shaped cross section, a longitudinal axis in the main channel is in line with a longitudinal axis in the sample channel, a longitudinal axis in the external channel crosses with a longitudinal axis in the main channel, and all the longitudinal axes in the main channel, the sample channel and the one or more external channel are in the same plane.

9. The coaxial channel comprising a cylinder channel having a sawtooth-shaped cross section according to claim 7, wherein a part of the sample channel having a sawtooth-shaped cross section is tapered.

10. A method for manufacturing a microfiber having a sawtooth-shaped cross section using the coaxial channel comprising the cylinder channel having a sawtooth-shaped cross section having (A) a main channel, (B) a sample channel and (C) one or more external channel, wherein at least one of the main channel, the sample channel and the one or more external channel is a cylinder channel having a circular or oval cross section, a terminal end of the sample channel is connected to an initial end of the main channel, (i) the sample channel is tapered toward the terminal end portion or (ii) only the terminal end portion of the sample channel is tapered toward the portion connected with the main channel and the remaining portion is constant in size and shape of the cross section, with at least a part of the sample channel having a sawtooth-shaped cross section, and the one or more external channel is connected to a side of the main channel, which comprises:
(A) injecting a sample material into the sample channel; and
(B) injecting an external material into the external channel.

11. The method for manufacturing a microfiber having a sawtooth-shaped cross section according to claim 10, wherein the sample material is (i) a non-UV-curable material selected from PLGA, alginate, chitosan and collagen, (ii) a UV-curable material selected from 4-HBA, PNIPAAM, Norland Optical Adhesive (NOA) and PEG or (iii) a mixture thereof and the external material is a solution wherein (i) a first external material selected from calcium chloride, sodium chloride and a mixture thereof is dissolved in (ii) a second external material selected from water, cell culture, PBS and a mixture thereof.

12. A method for controlling the diameter of a microfiber having a sawtooth-shaped cross section manufactured using the coaxial channel comprising the cylinder channel having a sawtooth-shaped cross section having (A) a main channel, (B) a sample channel and (C) one or more external channel, wherein at least one of the main channel, the sample channel and the one or more external channel is a cylinder channel having a circular or oval cross section, a terminal end of the sample channel is connected to an initial end of the main channel, (i) the sample channel is tapered toward the terminal end portion or (ii) only the terminal end portion of the sample channel is tapered toward the portion connected with the main channel and the remaining portion is constant in size and shape of the cross section, with at least a part of the sample channel having a sawtooth-shaped cross section, and the one or more external channel is connected to a side of the main channel, which comprises controlling (i) the injection rate of the sample material into the sample channel and (ii) the injection rate of the external material into the external channel, wherein (i) the injection rate of the sample material into the sample channel is controlled in the range of 0.6-1.8 mL/h and (ii) the injection rate of the external material into the external channel is controlled in the range of 20-40 mL/h.

13. A method for manufacturing a microparticle having a sawtooth-shaped cross section using the coaxial channel comprising the cylinder channel having a sawtooth-shaped cross section having (A) a main channel, (B) a sample channel and (C) one or more external channel, wherein at least one of the main channel, the sample channel and the one or more external channel is a cylinder channel having a circular or oval cross section, a terminal end of the sample channel is connected to an initial end of the main channel, (i) the sample channel is tapered toward the terminal end portion or (ii) only the terminal end portion of the sample channel is tapered toward the portion connected with the main channel and the remaining portion is constant in size and shape of the cross section, with at least a part of the sample channel having a sawtooth-shaped cross section, and the one or more external channel is connected to a side of the main channel, which comprises:
(A) injecting a sample material into the sample channel; and
(B) injecting an external material into the external channel.

14. The method for manufacturing a microparticle having a sawtooth-shaped cross section according to claim 13, wherein the sample material is (i) a non-UV-curable material selected from PLGA, alginate, chitosan and collagen, (ii) a UV-curable material selected from 4-HBA, PNIPAAM, Norland Optical Adhesive (NOA) and PEG or (iii) a mixture thereof, the external material comprises (i) a first external material selected from calcium chloride and sodium chloride and (ii) a second external material selected from oleic acid, soybean oil, methanol and dodecane and the external material is prepared by: (a) preparing a first external material solution by dissolving the first external material in (iii) 2-methyl-1-propanol as a third external material; (b) preparing a mixture solution by mixing the first external material solution with the third external material; and (c) distilling the mixture solution.

15. A method for controlling the diameter of a microparticle having a sawtooth-shaped cross section manufactured using the coaxial channel comprising the cylinder channel having a sawtooth-shaped cross section having (A) a main channel, (B) a sample channel and (C) one or more external channel, wherein at least one of the main channel, the sample channel and the one or more external channel is a cylinder channel having a circular or oval cross section, a terminal end of the sample channel is connected to an initial end of the main channel, (i) the sample channel is tapered toward the terminal end portion or (ii) only the terminal end portion of the sample channel is tapered toward the portion connected with the main channel and the remaining portion is constant in size and shape of the cross section, with at least a part of the sample channel having a sawtooth-shaped cross section, and the one or more external channel is connected to a side of the main channel, which comprises controlling (i) the injection rate of the sample material into the sample channel and (ii) the injection rate of the external material into the external channel, wherein (i) the injection rate of the sample material into the sample channel is controlled in the range of 0.6-1.8 mL/h and (ii) the injection rate of the external material into the external channel is controlled in the range of 5-35 mL/h.

16. A microfiber for nerve reconstruction comprising a plurality of sawtooth grooves on a surface thereof along a longitudinal direction so as to have a sawtooth-shaped cross section, wherein nerve cells are cultured on the surface of the microfiber along the sawtooth grooves.

17. The microfiber for nerve reconstruction according to claim 16, wherein the sawtooth groove has a thickness of 3-15 μm and a width of 5-15 μm and the gap between the sawtooth grooves is 10-20 μm.

18. A method for culturing nerve cells, comprising contacting a microfiber comprising a plurality of sawtooth grooves on a surface thereof along a longitudinal direction so as to have a sawtooth-shaped cross section with a culture medium containing nerve cells, wherein the nerve cells are cultured on the surface of the microfiber along the sawtooth grooves.

19. The method for culturing nerve cells according to claim 18, wherein the sawtooth groove has a thickness of 3-15 μm and a width of 5-15 μm and the gap between the sawtooth grooves is 10-20 μm.
